SEQUENCE LISTING (4 sequences)

SEQ ID NO: 1 - protein, 6 aa, Artificial Sequence
  Synthetic Peptide
VGPSQG

SEQ ID NO: 2 - protein, 6 aa, Artificial Sequence
  Synthetic Peptide
VGGSQG

SEQ ID NO: 3 - protein, 6 aa, Artificial Sequence
  Synthetic Peptide
VEPLQA

SEQ ID NO: 4 - protein, 6 aa, Artificial Sequence
  Synthetic Peptide
  MISC_FEATURE (2)..(2): Xaa is D-amino acid D-Ala
VXPSQG

(19) United States
(12) Patent Application Publication
(10) Pub. No.: US 2020/0055896 A1
(43) Pub. Date: Feb. 20, 2020

(54) SUBSTRATES RECOGNIZED BY FIBROBLAST ACTIVATION PROTEIN (FAP) AND METHODS OF USING THE SAME

(71) Applicant: GENENTECH, INC., South San Francisco, CA (US)

(72) Inventors: Travis William BAINBRIDGE, South San Francisco, CA (US); James Andrew ERNST, South San Francisco, CA (US); Junichiro SONODA, South San Francisco, CA (US)

(21) Appl. No.: 16/589,740

(22) Filed: Oct. 1, 2019

Related U.S. Application Data
(63) Continuation of application No. PCT/US2018/026009, filed on Apr. 4, 2018.
(60) Provisional application No. 62/481,539, filed on Apr. 4, 2017.

(51) Int. Cl. C07K 7/06; C07K 16/40; G01N 33/573; G01N 33/542; A61K 49/00; A61K 47/68; C12Q 1/37

(52) U.S. Cl. CPC C07K 7/06; C07K 2319/50; G01N 33/573; G01N 33/542; A61K 49/0056; A61K 49/0054; A61K 47/6871; C12Q 1/37; G01N 2800/7052; C07K 2319/30; G01N 2800/085; G01N 2800/042; G01N 2800/36; C07K 2317/31; C07K 16/40

(57) ABSTRACT

Fluorescence resonance energy transfer (FRET) constructs comprising donor and acceptor fluorophore moieties, and a peptide linking the two, which is a substrate of the endopeptidase fibroblast activation protein (FAP). Also provided are isolated nucleic acids expressing the construct, cell lines comprising the nucleic acids, and kits comprising the construct. Further provided are methods of detecting FAP using the construct via FRET.

### BACKGROUND

Human Fibroblast Activation Protein (FAP; GenBank Accession Number AAC51668; EC 3.4.21.B28), also known as FAPα, seprase, or circulating antiplasmin-cleaving enzyme, is a 170 kDa, type II transmembrane serine protease and a member of the S9 family of proline-specific proteases which includes dipeptidyl peptidase IV (DPPIV), DPP8, DPP9, prolyl endopeptidase (PREP, also known as POP). FAP is a homodimer containing two N-glycosylated subunits with a large C-terminal extracellular domain, in which the enzyme's catalytic domain is located. In its glycosylated form, FAP has both post-prolyl dipeptidyl peptidase and post-prolyl endopeptidase activities.

Human FAP was originally identified in cultured fibroblasts, and homologues of the protein were found in several species, including mice. Despite intense studies, the identities of physiological substrates for FAP remain elusive. FAP, like its closest relative, DPPIV, exhibits dipeptidyl peptidase activity, cleaving after the proline present at the second position from the N-terminus in various secreted proteins and peptide hormones. In addition, FAP, but not DPPIV, possesses endopeptidase activity, preferentially cleaving carboxy-terminal to a Gly-Pro sequence. While collagens have repeating Gly-Pro motifs, they are resistant to degradation by FAP except in a denatured or partially processed form (e.g., gelatin). Consistent with the role of FAP in regulating collagen turnover, Fap KO mice show elevated collagen levels in pathogenic conditions. FAP has also been proposed to cleave at a specific site near the N-terminus of α2-Antiplasmin (α2AP) to potentiate its activity. Additionally, the present inventors have found that FAP cleaves fibroblast growth factor 21 (FGF21) at a specific site proximal to the C-terminus thereby inactivating this metabolic hormone. In both α2AP and FGF21, the specific cleavage site targeted by FAP possesses the consensus Gly-Pro sequence at P2-P1 position, and these amino acid residues are essential for cleavage by FAP.

Although FAP is produced as a membrane-bound protein, the extracellular domain encoding the active enzyme can be shed from the cell surface, and therefore soluble FAP protein is readily detectable in serum and plasma by a standard sandwich ELISA. The level of FAP protein has been shown to be elevated in some pathological conditions. FAP has a unique tissue distribution: its expression was found to be highly upregulated in patients with cirrhosis and on reactive stromal fibroblasts of more than 90% of all primary and metastatic epithelial tumors, including lung, colorectal, bladder, ovarian and breast carcinomas, while it is generally absent from normal adult tissues. Subsequent reports showed that FAP is not only expressed in stromal fibroblasts but also in some types of malignant cells of epithelial origin, and that FAP expression directly correlates with the malignant phenotype.

Due to its expression in cirrhosis and many common cancers, and its restricted expression in normal tissues, FAP may be a promising target for imaging, diagnosis, and therapy of a variety of mammalian diseases. Thus, many monoclonal antibodies have been raised against FAP. Alternative, and less cumbersome approaches to measuring FAP protein levels and enzymatic activity are desired for research, diagnostic, and therapeutic purposes.

### SUMMARY

The present inventors have produced a homogeneous fluorescence intensity assay for circulating Fibroblast Activation Protein (FAP) endopeptidase activity that utilizes a modified peptide substrate based on the endopeptidase cleavage site of FGF21, a newly identified natural substrate for FAP, that is selective for FAP. This assay therefore relies on enzymatic activity to measure FAP protein levels, in contrast to the activity-independent ELISA.

In one aspect, this disclosure therefore provides a molecular fluorescence resonance energy transfer (FRET) construct comprising a linker peptide, a donor fluorophore moiety and an acceptor fluorophore moiety, wherein the linker peptide is a substrate of FAP, and is not a substrate for another human S9 peptidase, a human S28 peptidase, or any one of dipeptidyl peptidase IV (DPPIV), DPP8, or DPP9. Specifically, the FRET constructs of this disclosure are not substrates for prolyl endopeptidase (PREP).

The linker peptide of these FRET constructs separates the donor and acceptor fluorophores by a distance of not more than 10 nm and the absorption spectrum of the donor fluorophore moiety overlaps the excitation spectrum of the acceptor fluorophore moiety, such that specific cleavage by FAP causes sufficient separation of the donor and acceptor fluorophores to create a detectable change in fluorescence. Thus, the FRET constructs of this disclosure may be a quenched-FRET construct or a normal FRET construct, leading to either a decrease or an increase in fluorescence, respectively, following cleavage of the linker peptide by FAP enzymatic activity. A preferred fluorophore pair for this disclosure is FRET donor HyLite Fluor 488 and FRET acceptor QXL 520.

The linker peptide of these FRET constructs may comprise at least about 6 amino acid residues and an amino acid sequence selected from the group consisting of SEQ ID NOs: 1-4. These linker peptides may comprise at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22 amino acid residues, including a sequence selected from the group consisting of SEQ ID NOs:1-4.

These linker peptides may also comprise longer amino acid sequences, including sequences of at least about 30 amino acid residues, or at least about 40 amino acid residues, or at least about 45 amino acid residues, or at least about 50 amino acid residues, or at least about 55 amino acid residues, or at least about 65 amino acid residues, including a sequence selected from the group consisting of SEQ ID NOs:1-4.

This disclosure further provides an isolated polynucleotide molecule encoding a linker peptide as described above. The construct is preferably an expression vector comprising the polynucleotide molecule operably linked to a promoter. A preferable promoter for these expression vectors is an inducible promoter. This disclosure also provides a cell comprising one or more of these isolated polynucleotide molecules. The cell may be selected from a CHO cell, an E. coli cell, or a yeast cell.

This disclosure also provides a kit that comprises a FRET construct of this disclosure in a suitable container.

This disclosure also provides a method of detecting a FAP endopeptidase and/or FAP enzymatic activity, that includes providing a FRET construct described above, wherein the linker is a FAP substrate, exposing the construct to a sample suspected of containing FAP enzymatic activity under conditions under which FAP enzyme cleaves the FAP substrate, and detecting and comparing the FRET signal before and after the construct is exposed to the sample, wherein an increase in fluorescence indicates the presence of active FAP protein in the sample. In these methods, the linker peptide of the FRET construct may comprise the amino acid sequence of SEQ ID NO:4. In these methods, FRET may be detected by a method selected from measuring fluorescence emitted at the acceptor emission wavelength and donor emission wavelength, and determining energy transfer by the ratio of the respective emission amplitudes; measuring fluorescence lifetime of the donor; measuring the photobleaching rate of the donor; measuring anisotropy of the donor and acceptor; and measuring the Stokes shift monomer/excimer fluorescence.

This disclosure also provides methods for screening for an inhibitor of FAP endopeptidase activity, including providing a cell containing a FRET construct as described above, wherein the linker in the construct is a peptide substrate of FAP; exposing the cell to a candidate inhibitor compound; and detecting FRET signals of the cell before and after the exposure to inhibitor compound, wherein an observation of substantial decrease or elimination of FRET in the presence of the candidate inhibitor indicates that the candidate inhibitor is capable of inhibiting FAP. Preferably, the candidate compound is among a library of compounds and the method is a high throughput method.

Similarly, this disclosure provides methods for screening for an inhibitor of FAP endopeptidase activity, including providing an isolated FRET construct, substantially free of cellular components, as described above, wherein the linker in the construct is a peptide substrate of FAP; exposing the FRET construct to a candidate inhibitor compound; and detecting FRET signals before and after the exposure to inhibitor compound, wherein an observation of substantial decrease or elimination of FRET in the presence of the candidate inhibitor indicates that the candidate inhibitor is capable of inhibiting FAP. In these methods, the isolated FRET construct may be associated with (i.e., covalently or non-covalently linked to) a solid support that does not interfere with the fluorescence activity or the cleavage of the linker peptide by FAP. Preferably, the candidate compound is among a library of compounds and the method is a high throughput method.

This disclosure also provides methods for detecting FAP enzymatic activity, including a) providing a FRET construct as described above, b) exposing the construct to a sample suspected of containing FAP endopeptidase under a condition under which the FAP enzyme cleaves the linker portion of the FRET construct, and c) detecting spatial separation of the fluorescence signals of the donor and acceptor fluorophores, wherein occurrence of spatial separation indicates the presence of FAP enzymatic activity in the sample. Preferably, the construct is inside a live cell or tissue, the linker peptide is SEQ ID NO:4 linked to FRET donor HyLite Fluor 488 and FRET acceptor QXL 520, wherein detection of fluorescence indicates the existence of presence of FAP enzymatic activity in the sample.

This disclosure also provides methods of detecting or imaging the FRET construct in a subject. In one method, in vivo imaging is conducted when FAP-expressing tissue is imaged in a subject by administering to the subject a FRET construct of this disclosure, and detecting fluorescence from the molecular construct by in vivo imaging. In these methods, the FRET construct may comprise near-infrared fluorescence (NIRF) fluorophores. The in vivo imaging may be selected from NIRF imaging, fluorescence reflectance imaging (FRI), fluorescence-mediated tomography (FMT), and combinations thereof.

In another method, ex vivo imaging is conducted when a FAP-expressing tissue is imaged by obtaining a tissue sample from a subject; contacting the tissue sample with a FRET construct of this disclosure, and detecting fluorescence from the molecular construct by ex vivo imaging. The ex vivo imaging may comprise low resolution imaging with excised tissues. The ex vivo imaging may comprise in situ zymography of tissue slices obtained from the subject.

These methods may further include removing tissue (i.e., resecting tissue) from the subject that is detected or imaged, such as cancerous tissue, a solid tumor, a fibrotic tissue within an organ, or another part of an organ, after in vivo imaging of the subject. Surgical resection can be performed by any technique known in the art. In some embodiments, the method may further include administering the FRET construct after resection to measure the completeness of tissue resection.

This disclosure also provides methods of diagnosing, detecting, or monitoring fibrosis or cancer in a subject including contacting a test sample taken from the subject with a FRET construct of this disclosure, measuring the amount of fluorescence in the test sample; and normalizing the results against fluorescence from a control sample, such that the detection of fluorescence in the test sample is diagnostic of fibrosis or associated disorders, such as cancer, in the subject from which the sample was obtained. These methods may include administering to the subject a FRET construct of this disclosure, and thereafter obtaining the test sample from the subject, measuring the amount of fluorescence in the test sample; and normalizing the results against fluorescence from a control sample, such that the detection of fluorescence in the test sample is diagnostic of fibrosis or associated disorders, such as cancer, in the subject from which the sample was obtained.

This disclosure also provides kits for diagnosing, detecting, or monitoring fibrosis or associated disorders, such as cancer. These kits may include a FRET construct of this disclosure and instructions for their use.

The invention is described in more details below with the help of the drawings and examples, which are not to be construed to be limiting the scope of this disclosure.

### DESCRIPTION OF THE FIGURES

FIG. 1. Design of fluorescence-quenched peptides containing an N-terminal fluorescent donor, followed by six amino acid residues of the region flanking the dominant FAP endopeptidase cleavage site of human FGF21, ending with an additional C-terminal lysine conjugated to fluorescence acceptor. Variants (table) include substitution of the P1 proline with glycine, the P2 glycine with D-alanine or the entire homologous region of murine FGF21.

FIGS. 2A-2D show plasma cleavage of fluorescence-quenched peptides. FIG. 2A shows the cleavage rate of human WT (GP), human mutant (GG) and murine (EP) FGF21-based peptides in plasma samples isolated from Fap^{+/+}, Fap^{+/-}, and Fap^{-/-} mice, as indicated (N=3 mice per genotype). As a control, the cleavage reaction was run with 1 nM recombinant mouse FAP. Concentration of peptide substrates in this experiment was 3 μM. FIG. 2B shows the cleavage rate of the GP peptide by recombinant human FAP or PREP proteins at 1 nM enzyme concentration. FIG. 2C shows the cleavage rate of the GP peptide by recombinant human FAP (left, blue) or PREP (right, red) in the presence of a FAP-specific inhibitor cpd60 and/or PREP-specific inhibitor KYP-2047 (KYP). FIG. 2D shows the cleavage rate of the GP peptide in plasma from Fap^{+/+} (blue), Fap^{+/-} (red), and Fap^{-/-} (black) mice in the presence of cpd60 and/or KYP (N=3 per group).

FIGS. 3A-3D show cleavage kinetics of GP and aP substrates by recombinant FAP. FIGS. 3A and 3B show Michaelis-Menten saturation curves for human (black) or mouse (blue) recombinant FAP cleavage of (FIG. 3A) the GP or (FIG. 3B) aP peptides. FIG. 3C is a plot of fractional velocity as a function of cpd60 concentration, for recombinant mouse or human FAP with cpd60, using GP peptide. Mouse and human FAP preparations were found to be 74% and 57% active, respectively. FIG. 3D shows human and mouse FAP enzyme kinetics constants with the GP and aP substrates, corrected for experimentally determined active enzyme concentrations determined in FIG. 3C. Error is SEM, determined from three experimental replicates. FAP molar concentrations are based on monomer for active site titration and kinetics calculations.

FIGS. 4A-4F shows the specificity of aP peptide cleavage. FIG. 4A shows the cleavage rate of the aP or GG peptide by 1 nM recombinant, human FAP or PREP. FIG. 4B shows the cleavage rate of the aP peptide by a panel of recombinant, human prolyl peptidases. Each recombinant peptidase was used at 2.5 μg/ml. FIGS. 4C and 4D show the cleavage rate of ANP_{FAP} (C) or aP NIRF (D) by 10 nM recombinant, human FAP or PREP. FIG. 4E shows the cleavage rate of the aP peptide in plasma from Fap^{+/+}, Fap^{-/-}, and Fap^{-/-} mice. Plasma was diluted 10-fold. FIG. 4F shows the cleavage rate of the aP peptide in anti-FAP or control IgG-immunodepleted human serum. Serum was diluted 2.5-fold. Complete FAP removal following anti-FAP immunodepletion was shown previously by immunoblotting (Dunshee, et al. (2016) J Biol Chem 291:5986-96).

FIGS. 5A-5C show that FAP activity in serum correlates with liver disease and FAP levels. FIG. 5A is a scatter plot showing FAP activity levels determined by using the aP peptide (x axis) and FAP protein levels determined by ELISA in serum samples (y axis) from healthy individuals (N=16, black circles) or individuals previously diagnosed with liver cirrhosis (N=17, red circles). R² value is indicated in the graph. FIGS. 5B and 5C show the same data as FIG. 5A presented as mean±SEM. P values indicated were calculated by student t-test.

FIG. 6 shows that plasma FAP activity is completely suppressed in mice fed chow with cpd60. Ten to eighteen-week old C57/BL6 background mice were fed ad libitum control chow or diet containing 100 ppm of compound 60. Plasma was isolated from blood retrieved at the indicated time points and FAP activity levels determined using the aP peptide. N=6 animals per group (2 males and 4 females for the control group, 3 females and 3 males for the cpd 60 diet group). P values indicated were calculated by student t-test (****: P<0.0001; **: P<0.002).

FIG. 7A shows ex vivo images of three pancreata. The gross morphology is shown in the left panel, and a heatmap representation of an aP NIRF probe imaging signal intensity is shown in the right panel. FIG. 7B shows quantification of relative fluorescence intensities normalized to imaging areas indicating greater fluorescence in KPP pancreata.

### DETAILED DESCRIPTION

This disclosure provides novel compositions and methods based on fluorescence resonance energy transfer (FRET) between fluorophores linked by a peptide linker which is a substrate of, and is cleaved by, Fibroblast Activation Protein (FAP), to detect and monitor FAP enzymatic activity. These method and compositions allow for the detection of picomolar level FAP and are useful in high-throughput assay systems for large-scale screening of FAP inhibitors. These compositions and methods are also useful for imaging FAP activity in living cells, tissues, and organs, as well as ex vivo imaging of such tissues.

Fluorescence Resonance Energy Transfer (FRET) is a tool that allows the assessment of the distance between one molecule and another (e.g. a protein or nucleic acid) or between two positions on the same molecule. FRET is known in the art (for reviews, see Matyus, (1992) J. Photochem. Photobiol. B: Biol., 12:323; and olympusmicro.com/primer/techniques/fluorescence/fret/fretintro.html). FRET is a radiation-less process in which energy is transferred from an excited donor molecule to an acceptor molecule. Radiation-less energy transfer is the quantum-mechanical process by which the energy of the excited state of one fluorophore is transferred without actual photon emission to a second fluorophore. Briefly, a fluorophore absorbs light energy at a characteristic wavelength. This wavelength is also known as the excitation wavelength. The energy absorbed by a fluorochrome is subsequently released through various pathways, one being emission of photons to produce fluorescence. The wavelength of light being emitted is known as the emission wavelength and is an inherent characteristic of a particular fluorophore. In FRET, that energy is released at the emission wavelength of the second fluorophore. The first fluorophore is generally termed the donor and has an excited state of higher energy than that of the second fluorophore, termed the acceptor. An essential feature of the process is that the emission spectrum of the donor overlap with the excitation spectrum of the acceptor, and that the donor and acceptor be sufficiently close. The distance between the donor and the acceptor must be sufficiently small to allow the radiation-less transfer of energy between the fluorophores. Because the rate of energy transfer is inversely proportional to the sixth power of the distance between the donor and acceptor, the energy transfer efficiency is extremely sensitive to distance changes. Energy transfer is said to occur with detectable efficiency in the 1-10 nm distance range, but is typically 4-6 nm for optimal results. The distance range over which radiation-less energy transfer is effective depends on many other factors as well, including the fluorescence quantum efficiency of the donor, the extinction coefficient of the acceptor, the degree of overlap of their respective spectra, the refractive index of the medium, and the relative orientation of the transition moments of the two fluorophores.

FRET Constructs of this Disclosure

This disclosure provides a construct (“FRET construct”) which comprises a fluorophore FRET donor and an acceptor linked by linker peptide (“substrate peptide”) that is cleavable by FAP. In the presence of FAP enzymatic activity, the linker peptide is cleaved, thereby leading to a decrease in energy transfer and increased emission of light by the donor fluorophore or decreased emission of light by the acceptor. In this way, the proteolysis activity of FAP may be detected, monitored, and quantitated.

In these FRET constructs, the linker peptide preferably is not a substrate for a human S9 peptidase or a human S28 peptidase. Similarly, the linker peptide is preferably not a substrate for any one of dipeptidyl peptidase IV (DPPIV), DPP8, or DPP9. Even more preferably, the linker peptide is not a substrate for prolyl endopeptidase (PREP).

In these FRET constructs, the linker peptide separates the donor and acceptor fluorophores by a distance of not more than 10 nm, and the emission spectrum of the donor fluorophore moiety overlaps with the absorption spectrum of the acceptor fluorophore moiety.

Exemplary FRET constructs of this disclosure include molecular constructs comprising the sequence:

| P3 | P2 | P1 | P1′ | P2′ | P3′ | |
|---|---|---|---|---|---|---|
| Xaa1- | Xaa2- | Xaa3- | Xaa4- | Xaa5- | Xaa6- | (SEQ ID |
| | | | | | | NO: 1) |

wherein

Xaa1 is: a natural or non-natural amino acid residue or derivative;

Xaa2 is: a residue selected from the D-enantiomer of a naturally occurring amino acid, or an amino acid analog;

Xaa3 is: proline (Pro) or a derivative thereof;

Xaa4 is: a natural or non-natural amino acid residue or derivative;

Xaa5 is: a natural or non-natural amino acid residue or derivative; and,

Xaa6 is: a natural or non-natural amino acid residue or derivative.

In these exemplary FRET constructs, Xaa2 may be selected from D-alanine, D-serine, and D-threonine, or Xaa2 may be an amino acid analog having the formula —NH—(CH2)n-COO— wherein n=2-10. Xaa2 may therefore be an amino acid analog selected from beta-alanine (bAla), gamma-aminobutryic acid (4Abu), 5-aminovaleric acid, and 6-aminohexanoic acid.

In these exemplary FRET constructs, Xaa3 may be a halogenated proline residue. The molecular construct of claim 11, such as Xaa3 a fluorinated proline residue. Xaa3 may also be a derivative of proline selected from dehydroproline, 4,4-difluoroproline, 3-fluroproline, 4-fluroproline, 3-hydroxyproline (3Hyp), and 4-hydroxyproline (4Hyp).

In these exemplary FRET constructs, Xaa1 may be covalently linked to either the donor fluorophore moiety or the acceptor fluorophore moiety. Similarly, in these constructs, Xaa6 may be covalently linked to either the donor fluorophore moiety or the acceptor fluorophore moiety. At least one of the donor fluorophore moiety and the acceptor fluorophore moiety may be covalently linked to the linker peptide through a linker. If a linker is present, the linker may comprise at least one amino acid selected from lysine (Lys), arginine (Arg), glutamine (Gln), and asparagine (Asn). In exemplary constructs, the linker is a lysine (Lys) residue. The donor fluorophore moiety or the acceptor fluorophore moiety may be linked to the amino-terminus of the linker peptide. Alternatively or additionally, the donor fluorophore moiety or the acceptor fluorophore moiety may be linked to the carboxy-terminus of the peptide. Thus, in one configuration, the donor fluorophore moiety is linked to the carboxy-terminus of the peptide, and the acceptor fluorophore moiety is linked to the amino-terminus of the peptide. In another configuration, the donor fluorophore moiety is linked to the amino-terminus of the peptide and the acceptor fluorophore moiety is linked to the carboxy-terminus of the peptide.

An exemplary linker peptide of this disclosure comprises or consists of a peptide having the sequence Val-(D-Ala)-Pro-Ser-Gln-Gly (SEQ ID NO:2), or a peptide comprising at least 80% amino acid sequence identity to the amino acid sequence of SEQ ID NO:2, while remaining a substrate for FAP but not a substrate for another human S9 or S28 peptidase. Similarly, the linker peptide may be a peptide with at least 80% amino acid sequence identity to the amino acid sequence of SEQ ID NO:2, while remaining a substrate for FAP but not a substrate for a human S9 or S28 peptidase.

An exemplary linker peptide of this disclosure may comprise or consist of a peptide having between 1 and 3 conservative amino acid substitutions compared to the amino acid sequence of SEQ ID NO:2, while remaining a substrate for FAP but not a substrate for a human S9 or S28 peptidase. Similarly, the linker peptide may be a peptide comprising one conservative amino acid substitution compared to the amino acid sequence of SEQ ID NO:2, while remaining a substrate for FAP but not a substrate for a human S9 or S28 peptidase.

Also contemplated in the context of the inventive methods and compositions of this disclosure is the modification of any linker peptides, by chemical or genetic means. Examples of such modification include construction of peptides of partial or complete sequence with non-natural amino acids and/or natural amino acids in L- or D-enantiomeric forms. For example, any of the linker peptides disclosed herein, and any variants thereof, could be produced in an all-D form. Furthermore, the polypeptides may be modified to contain carbohydrate or lipid moieties, such as sugars or fatty acids, covalently linked to the side chains or the N- or C-termini of the amino acids. In addition, the linker peptides of this disclosure may also comprise a modification selected from phosphorylation and glycosylation.

In addition, the linker peptides of this disclosure may be modified to enhance solubility and/or half-life upon being administered. For example, polyethylene glycol (PEG) and related polymers have been used to enhance solubility and the half-life of protein therapeutics in the blood. Accordingly, the linker peptides of this disclosure may be modified by PEG polymers and the like. PEG or PEG polymers means a residue containing poly(ethylene glycol) as an essential part. Such a PEG can contain further chemical groups which are necessary for the therapeutic activity of the linker peptides of this disclosure; which results from the chemical synthesis of the molecule; or which is a spacer for optimal distance of the parts of the molecule from one another. In addition, such a PEG can consist of one or more PEG side-chains which are linked together. PEG groups with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEGs usually have 2 to 8 arms and are described in, for example, U.S. Pat. No. 5,932,462. Especially preferred are PEGs with two PEG side-chains (PEG2) linked via the primary amino groups of a lysine (Monfardini, C, et al., Bioconjugate Chem. 6 (1995) 62-69). The term “PEG” is used broadly to encompass any polyethylene glycol molecule, wherein the number of ethylene glycol (EG) units is at least 460, preferably 460 to 2300 and especially preferably 460 to 1840 (230 EG units refers to a molecular weight of about 10 kDa). The upper number of EG units is only limited by solubility of the PEGylated linker peptides of this disclosure. Usually PEGs which are larger than PEGs containing 2300 units are not used. Preferably, a PEG used in the invention terminates on one end with hydroxy or methoxy (methoxy PEG, mPEG) and is on the other end covalently attached to a linker moiety via an ether oxygen bond. The polymer is either linear or branched. Branched PEGs are e.g. described in Veronese, F. M., et al., Journal of Bioactive and Compatible Polymers 12 (1997) 196-207. Suitable processes and preferred reagents for the production of PEGylated linker peptides and variants of this disclosure are described in US Patent Pub. No. 2006/0154865. It is understood that modifications, for example, based on the methods described by Veronese, F. M., Biomaterials 22 (2001) 405-417, can be made in the procedures so long as the process results in PEGylated linker peptides of this disclosure. Particularly preferred processes for the preparation of PEGylated linker peptides of this disclosure are described in US 2008/0119409, which is incorporated herein by reference. Exemplary linker peptides of this disclosure may be linked to a polyethylene glycol (PEG) molecule, such as a PEG molecule comprising between 75 and 2000 ethylene glycol (EG) units.

Additionally or alternatively, the linker peptides of this disclosure may be is fused to one or more domains of an Fc region of human IgG. Antibodies comprise two functionally independent parts, a variable domain known as “Fab,” that binds an antigen, and a constant domain known as “Fc,” that is involved in effector functions such as complement activation and attack by phagocytic cells. An Fc has a long serum half-life, whereas a Fab is short-lived (Capon et al., 1989, Nature 337:525-31). When constructed together with a therapeutic protein of this disclosure, an Fc domain can provide longer half-life or incorporate such functions as Fc receptor binding, protein A binding, complement fixation, and perhaps even blood-brain barrier, or placental transfer. In one example, a human IgG hinge, CH2, and CH3 region may be fused at either the amino-terminus or carboxyl-terminus of the linker peptides of this disclosure using methods known to the skilled artisan. The resulting fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region may be a naturally occurring Fc region, or may be altered to improve certain qualities, such as therapeutic qualities, circulation time, or reduced aggregation. Exemplary linker peptides may be linked to one or more domains of an Fc region of human IgG molecule, such as a human IgG hinge, CH2, and/or CH3 region, that is fused to at least one of the amino-terminus or carboxyl-terminus of the peptide.

The linker peptides may also be modified to contain sulfur, phosphorous, halogens, metals, etc. Amino acid mimics may be used to produce polypeptides, and therefore, the linker peptides of this disclosure may include amino acid mimics that have enhanced properties, such as resistance to degradation. For example, the linker peptides may include one or more (e.g., all) peptide monomers.

The linker peptides of this disclosure may include “epitope tagged” peptides, which refers to a chimeric peptide comprising a linker peptide of this disclosure fused to a “tag polypeptide.” The tag polypeptide has enough residues to provide an epitope against which an antibody can be made, yet is short enough such that it does not interfere with activity of the inhibitory polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

The linker peptides of this disclosure may also be linked to, or associated with, a “solid phase” or “solid support” which is a non-aqueous matrix to which a peptide of this disclosure can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. Depending on the context, the solid phase can comprise the well of an assay plate or a purification column (e.g., an affinity chromatography column). This term also includes a discontinuous solid phase of discrete particles, such as those described in U.S. Pat. No. 4,275,149. Thus, the linker peptides of this disclosure may also be linked to a solid support, such as a solid support comprising at least one of glass, polysaccharides, polyacrylamides, polystyrene, polyvinyl alcohol, and silicones.

This disclosure also contemplates any of the substrate peptides described herein with any FRET pair on the termini or within the sequences of SEQ ID NOs:1-4, preferably SEQ ID NO: 4. For example, one common pair of fluorophores for biological use is a cyan fluorescent protein (CFP)-yellow fluorescent protein (YFP) pair. In the absence of FAP enzymatic activity, the reporter is intact and the CFP and YFP moieties are in close proximity. Excitation of CFP results in energy transfer to YFP due to FRET. As a consequence, CFP emission is quenched while YFP emits fluorescence due to FRET. In the presence of FAP enzymatic activity, the reporter is cleaved by the proteolytic activity of FAP. The CFP and YFP moieties are physically separated and FRET can no longer occur. CFP emission is restored and YFP emission is reduced. Adding FRET tags into the peptide substrates disclosed herein should generate higher detection than the FRET substrates that are commercially available. The peptide substrates of this disclosure advantageously have a low limit of detection of FAP enzymatic activity.

As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radioactive energy transfer can be produced.

As used herein with respect to substrate peptide and FAP, “corresponding” refers to a FAP endopeptidase that cleaves the linker peptide at a specific cleavage site.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

A skilled artisan will recognize that many fluorophore molecules are suitable for FRET. Fluorescent proteins may be used as fluorophores. Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyana tostilbene-2,2′-disulfonic acid, 7 diethylamino-3-(4′-isothiocyanatophenyl)-4-methylcoumarin, succinimdyl 1-pyrenebutyrate, and 4-acetamido-4′-isothiocyanatostilbene-2-2,2′-disulfonic acid derivatives. Representative acceptor fluorescent moieties, depending upon the donor fluorescent moiety used, include LC-Red 640, LC-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

Examples of fluorescence labels for use in the FRET constructs of this disclosure include fluorescein, 6-FAM™ (Applied Biosystems, Carlsbad, Calif.), TET™ (Applied Biosystems, Carlsbad, Calif.), VIC™ (Applied Biosystems, Carlsbad, Calif.), MAX, HEX™ (Applied Biosystems, Carlsbad, Calif.), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rho101, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, TAMRA™ (Applied Biosystems, Carlsbad, Calif.), 5-TAMRA, ROX™ (Applied Biosystems, Carlsbad, Calif.), Oregon Green® (Life Technologies, Grand Island, N.Y.), Oregon Green 500, IRDye® 700 (Li-Cor Biosciences, Lincoln, Nebr.), IRDye 800, WellIRED D2, WellIRED 5 D3, WellIRED D4, and Lightcycler® 640 (Roche Diagnostics GmbH, Mannheim, Germany).

Suitable acceptors include Black Hole Quencher®-1 (Biosearch Technologies, Novato, Calif.), BHQ-2, Dabcyl, Iowa Black® FQ (Integrated DNA Technologies, Coralville, Iowa), lowaBlack RQ, QXL™ (AnaSpec, Fremont, Calif.), QSY 7, QSY 9, QSY 21, QSY 35, and IRDye QC.

In addition to the linker peptides (“substrate peptides”) described herein, it is contemplated that linker peptide variants can be prepared. Linker peptide variants can be prepared by introducing appropriate nucleotide changes into the encoding DNA, and/or by synthesis of the desired polypeptide.

Variations in the linker peptide described herein can be made, for example, using any of the techniques and guidelines for conservative and non-conservative mutations set forth, for instance, in U.S. Pat. No. 5,364,934. Variations may be a substitution, deletion, or insertion of one or more codons encoding the linker peptide that results in a change in the amino acid sequence as compared with the native sequence polypeptide. Optionally, the variation is by substitution of at least one amino acid with any other amino acid. Guidance in determining which amino acid residue may be inserted, substituted, or deleted without adversely affecting the desired activity may be found by comparing the sequence of the linker peptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions, or substitutions of amino acids in the sequence and testing the resulting variants for activity exhibited by the full-length or mature native sequence.

Linker peptide fragments are provided herein. Such fragments may be truncated at the N-terminus or C-terminus, or may lack internal residues, for example, when compared with a full length native peptide. Certain fragments lack amino acid residues that are not essential for the detection of FAP enzymatic activity.

Linker peptides and fragments may be prepared by any of a number of conventional techniques. Desired peptide fragments may be chemically synthesized. An alternative approach involves generating polypeptides by enzymatic digestion, e.g., by treating a protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired peptide or fragment, by polymerase chain reaction (PCR). Oligonucleotides that define the desired termini of the DNA fragment are employed at the 5′ and 3′ primers in the PCR. Linker peptide fragments share at least partial FAP substrate specificity with the native linker peptide disclosed herein.

Conservative amino acid substitutions are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications to the linker peptides are accomplished by selecting substitutions that differ significantly in their effect on maintaining (a) the structure of the peptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, lie;
(2) neutral hydrophilic: Cys, Ser, Thr; Asn; Gin
(3) acidic: Asp, Glu;
(4) basic: His, Lys, Arg;
(5) residues that influence chain orientation: Gly, Pro; and
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the linker peptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-85 (1989)). Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isoteric amino acid can be used.

From the above discussion on the minimum cleavage sites and the relationship between FRET signal strength and linker length, and between cleavage efficiency and linker length, a person skilled in the art can easily choose suitable linker length to achieve optimal balance between FRET signal strength and cleavage efficiency.

Preferably, the linker length is anywhere between about 4 amino acids and about 100 amino acids, between 6-50 amino acids, between 7-30 amino acids, depending on the contemplated use of the FRET construct in detecting or imaging FAP enzymatic activity in a sample.

These linker peptides or fragments may be first purified, or peptides are first synthesized, and then the fluorescence groups are added onto certain amino acids through chemical reaction. A fluorescent label is either attached to the linker polypeptide or, alternatively, a fluorescent protein is fused in-frame with a linker polypeptide, as described below. Thus, while short substrate fragments are desirable for FAP enzymatic activity detection specificity, longer fragments may be desirable for improved signal strength or cleavage specificity, or to function as another part of a detection, imaging or diagnostic technology. In the case of a longer linker peptide, especially a full-length substrate protein, is used, the substrate may be engineered, e.g., via site-directed mutagenesis or other molecular engineering methods well-known to those skilled in the art, such that it contains only one FAP peptidase recognition site. Preferably, the linker peptides of this disclosure are designed using a combination of specificity engineering and length optimization to achieve optimal signal strength, cleavage efficiency and peptidase specificity.

The fluorophores are suitable fluorescent proteins linked by a suitable substrate peptide. A FRET construct may then be produced via the expression of recombinant nucleic acid molecules comprising an in-frame fusion of sequences encoding such a polypeptide and a fluorescent protein label either in vitro (e.g., using a cell-free transcription/translation system, or instead using cultured cells transformed or transfected using methods well known in the art).

Nucleic acid molecules encoding amino acid sequences of the linker peptides of this disclosure, or variants thereof, are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the linker peptide.

The nucleic acid (e.g., cDNA or genomic DNA) encoding the peptide linkers may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The linker peptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the linker peptide-encoding DNA that is inserted into the expression vector. The signal sequence may be a prokaryotic signal sequence selected, for example, from the group of the alkaline phosphatase, penicillinase, Ipp, or heat-stable enterotoxin II leaders. For yeast secretion, the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2p plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the linker peptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the linker peptide-encoding nucleic acid sequence to direct mRNA synthesis. Promoters recognized by a variety of potential host cells are well known. Promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980)), and hybrid promoters such as the tac promoter (deBoer et al., Proc. Natl. Acad. Sci. USA, 80:21-25 (1983)). Promoters for use in bacterial systems also will contain a Shine-Dalgarno sequence operably linked to the DNA encoding the linker peptide.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Linker peptide transcription from vectors in mammalian host cells is controlled, for example, by promoters obtained from the genomes of viruses such as polyoma virus, fowlpox virus (UK 2,211,504), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of DNA encoding the linker peptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the linker peptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding linker peptide.

Still other methods, vectors, and host cells suitable for adaptation to the synthesis of linker peptide in recombinant vertebrate cell culture are described in Gething et al., Nature, 293:620-25 (1981); Mantei et al., Nature, 281:40-46 (1979); EP 117,060; and EP 117,058.

Suitable cells for producing the FRET construct may be a bacterial, fungal, plant, or an animal cell. The FRET construct may also be produced in vivo, for example in a transgenic plant, or in a transgenic animal including, but not limited to, insects, amphibians, and mammals. A recombinant nucleic acid molecule of use in the invention may be constructed and expressed by molecular methods well known in the art, and may additionally comprise sequences including, but not limited to, those which encode a tag (e.g., a histidine tag) to enable easy purification, a linker, a secretion signal, a nuclear localization signal or other primary sequence signal capable of targeting the construct to a particular cellular location, if it is so desired.

Host cells are transfected or transformed with expression or cloning vectors described herein for linker peptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂), CaPO4, liposome-mediated, and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation; and an E. coli strain having mutant periplasmic protease disclosed in U.S. Pat. No. 4,946,783. Alternatively, in vitro methods of cloning, e.g., PCR or other nucleic acid polymerase reactions, are suitable.

Full length linker peptides, fragments thereof, or fusion proteins can be produced in bacteria, in particular when glycosylation of the linker peptides are not desired or needed. Production in E. coli is faster and more cost efficient. After expression, the linker peptide is isolated from the E. coli cell paste in a soluble fraction and can be purified. Final purification can be carried out similar to the process for purifying peptides expressed, e.g., in CHO cells.

In addition to prokaryotes, eukaryotic microbes such as filamentous fungi or yeast are suitable cloning or expression hosts for linker peptide-encoding vectors. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated peptides are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to this disclosure, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for peptide linker production and cultured in conventional nutrient media modified as appropriate for inducing promoters, selecting transformants, or amplifying the genes encoding the desired sequences.

Host cells used to produce the linker peptides of this disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Fluorescence signals sufficient to be detected using a microplate spectrofluorometer may be generated by as low as 300 nM of these FRET constructs of this disclosure. The fluorescence signal change can be traced in real time to reflect the FAP enzymatic activity. Real time monitoring measures signal changes as a reaction progresses, and allows both rapid data collection and yields information regarding reaction kinetics under various conditions. FRET ratio changes and degrees of cleavage may be correlated, for example for a certain spectrofluorometer using a method such as HPLC assay in order to correlate the unit of kinetic constant from the FRET ratio to substrate concentration.

The methods of this disclosure are highly sensitive, and as a consequence, can be used to detect trace amount of FAP enzyme or FAP enzymatic activity in environmental samples directly. Accordingly, this disclosure further provides a method for toxin detection and identification directly using environmental samples.

This disclosure further provides a method for screening for inhibitors of FAP using the above described in vitro systems. Because of its high sensitivity, rapid readout, and ease of use, in vitro systems based on this disclosure are also suitable for screening FAP inhibitors. Specifically, a FRET construct of this disclosure is exposed to FAP, in the presence of a candidate inhibitor substance, and changes in FRET signals are monitored to determine whether the candidate inhibits the activities of FAP.

In these methods of detecting FAP enzymatic activity or screening putative inhibitors, a cell-based system may be used in which a FRET construct of this disclosure is expressed inside a cell or on the surface of a cell, and the cell is then exposed to a sample suspected of containing FAP enzymatic activity. Changes in FRET signals are then monitored as an indication of the presence/absence or concentration of the FAP enzymatic activity. Specifically, an increase in FRET signals indicates that the sample contains FAP enzymatic activity.

Similarly, a cell expressing a FRET construct may be exposed to FAP in the presence of a candidate inhibitor substance, and changes in FRET signals are monitored to determine whether the candidate inhibits the enzymatic activity of FAP.

In these methods, fluorescent detection and analysis can be carried out using, for example, a photon counting epifluorescent microscope system (containing the appropriate dichroic mirror and filters for monitoring fluorescent emission at the particular range), a photon counting photomultiplier system, or a fluorometer. Excitation to initiate energy transfer can be carried out with an argon ion laser, a high intensity mercury (Hg) arc lamp, a fiber optic light source, or other high intensity light source appropriately filtered for excitation in the desired range. It will be apparent to those skilled in the art that excitation/detection means can be augmented by the incorporation of photomultiplier means to enhance detection sensitivity. For example, the two-photon cross correlation method may be used to achieve the detection on a single-molecule scale (see e.g., Kohl et al., Proc. Natl. Acad. Sci., 99:12161, 2002).

This disclosure also provides a composition comprising a FRET construct of this disclosure, and a carrier. For the purposes of imaging, diagnosing, and/or treating fibrosis or a fibrosis-related disease or disorder, these compositions may be administered to the patient in need of such treatment, wherein the composition can comprise one or more FRET constructs of this disclosure. In a further embodiment, these compositions may comprise the FRET construct in combination with other therapeutic agents. The formulation is therefore a therapeutic formulation comprising a pharmaceutically acceptable carrier.

Pharmaceutical formulations of a FRET construct of this disclosure may be prepared by mixing such construct having the desired degree of purity with one or more optional pharmaceutically acceptable carriers (Remington's Pharmaceutical Sciences 16th edition, Osol, A. Ed. (1980)), in the form of lyophilized formulations or aqueous solutions. Such pharmaceutical formulations a be useful for administration to a subject for diagnosis or imaging of cells or tissues having FAP enzymatic activity. Pharmaceutically acceptable carriers are generally nontoxic to recipients at the dosages and concentrations employed, and include, but are not limited to: buffers such as phosphate, citrate, acetate and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 can talk (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

This disclosure also provides assay devices, kits, and articles of manufacture comprising at least one FRET construct of this disclosure. The articles of manufacture may contain materials useful for the detection, imaging, diagnosis, or treatment of fibrosis or fibrosis-related diseases or disorders. A lateral flow assay device provides for point-of-care detection and/or diagnosis of fibrosis, particularly from blood or serum samples. The article of manufacture comprises a container and a label or package insert on or associated with the container. Suitable containers include, for example, bottles, vials, syringes, etc. The containers may be formed from a variety of materials such as glass or plastic. The container holds a composition which is effective for detecting, imaging, or treating fibrosis and may have a sterile access port (for example the container may be an intravenous solution bag or a vial having a stopper pierceable by a hypodermic injection needle). At least one active agent in the composition is FRET construct of this disclosure. The label or package insert indicates that the composition is used for detecting or treating fibrosis. The label or package insert may further comprise instructions for using the composition, e.g., in the testing or treating of the patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for fibrotic or tumor cell killing assays, for purification, or immunoprecipitation of FAP enzyme from cells. For isolation and purification of FAP protein, the kit can contain linker peptides coupled to beads (e.g., sepharose beads). Kits can be provided which contain the FRET constructs of this disclosure for detection and quantitation of FAP enzyme in vitro. As with the article of manufacture, the kit comprises a container and a label or package insert on or associated with the container. The container holds a composition comprising at least one FRET construct of this disclosure. Additional containers may be included that contain, e.g., diluents and buffers, or control enzymes or peptide constructs or FRET constructs. The label or package insert may provide a description of the composition as well as instructions for the intended diagnostic, imaging, detection, or therapeutic use.

The FRET construct of this disclosure may also be provided as part of an assay device. Such assay devices include lateral flow assay devices. A common type of disposable lateral flow assay device includes a zone or area for receiving the liquid sample, a conjugate zone, and a reaction zone. These assay devices are commonly known as lateral flow test strips. They employ a porous material, e.g., nitrocellulose, defining a path for fluid flow capable of supporting capillary flow. Examples include those described in U.S. Pat. Nos. 5,559,041, 5,714,389, 5,120,643, and 6,228,660 all of which are incorporated herein by reference in their entireties. The linker peptide or oligopeptide of this disclosure may also be used in a lateral flow assay device in conjunction with detect technologies using a single biological sample from a subject or patient being tested on one portable, point-of-care device.

Another type of assay device is a non-porous assay device having projections to induce capillary flow. Examples of such assay devices include the open lateral flow device as disclosed in PCT International Publication Nos. WO 2003/103835, WO 2005/089082, WO 2005/118139, and WO 2006/137785, all of which are incorporated herein by reference in their entireties.

The FRET constructs provided herein may be useful for detecting the presence of FAP enzymatic activity in a biological sample. The term “detecting” as used herein encompasses quantitative or qualitative detection. In certain embodiments, a biological sample comprises a cell or tissue, such as cells or tissues from brain, breast, colon, kidney, liver, lung, ovary, pancreas, prostate, skeletal muscle, skin, small intestine, stomach or uterus, including also cells or tissues tumors of these organs.

The disclosure therefore provides methods useful for treating fibrosis or fibrosis-related diseases or disorders, or alleviating one or more symptoms of fibrosis in a mammal, comprising administering a therapeutically effective amount of a therapeutic composition of this disclosure to the mammal. The therapeutic compositions can be administered short term (acutely) or chronically, or intermittently as directed by a medical professional. Also provided are methods of inhibiting the growth of, and killing a FAP enzyme-expressing cell.

Also contemplated is a FRET construct of this disclosure for use in a method of diagnosis or detection. In a further aspect, a method of detecting the presence of FAP enzymatic activity in a biological sample is provided. The method may include contacting a biological sample from a subject, optionally with a control sample, with FRET construct of this disclosure as described herein under conditions permissive for FAP enzyme cleavage of the linker peptide, and detecting florescence emitted by the FRET construct. Such method may be an in vitro or in vivo method. In one embodiment, a FRET construct of this disclosure is used to select subjects eligible for therapy with anti-fibrosis or anticancer therapies, e.g. where FAP enzymatic activity is a biomarker for selection of patients.

Exemplary disorders that may be diagnosed using a FRET construct of this disclosure include disorders associated with the expression of FAP or FAP enzymatic activity, such as fibrosis, cancer, and certain inflammatory conditions. In one aspect, a method of diagnosing disease in a subject is provided, the method comprising administering to the subject an effective amount of a diagnostic agent that comprises a FRET construct of this disclosure that allows detection of FAP enzymatic activity.

An exemplary diagnostic method of this disclosure includes obtaining a biological sample from a mammalian subject, detecting whether FAP enzymatic activity is present in the sample by contacting a portion of the sample with a FRET construct of this disclosure, illuminating the sample, detecting fluorescence resulting from FAP cleavage of the FRET construct; and diagnosing the subject with fibrosis-associated disease or disorder when fluorescence resulting from FAP enzymatic activity is detected in the sample.

Any of the FRET constructs or pharmaceutical formulations comprising the FRET constructs of this disclosure may also be used in therapeutic methods. An exemplary therapeutic method of this disclosure includes obtaining a biological sample from a human subject; detecting whether FAP enzymatic activity is present in the sample by contacting a portion of the sample with a FRET construct of this disclosure, illuminating the sample, detecting fluorescence resulting from FAP cleavage of the FRET construct, diagnosing the subject with a fibrosis-associated disease or disorder by comparing the fluorescence resulting from FAP cleavage of the molecular construct to a reference level of FAP enzymatic activity in the fibrosis-associated disease or disorder, wherein a statistically equal or higher level of FAP enzymatic activity in the sample compared to the reference level is indicative of a fibrosis-associated disease or disorder in the subject; and, administering an effective therapy to the diagnosed subject. Suitable therapies for use in these methods of treatment may include the administration of an inhibitor of FAP enzymatic activity, such as an anti-FAP enzyme antibody, which may include bi-specific antibodies, and/or antibody drug conjugates (ADCs).

The FRET constructs of this disclosure are useful for diagnosing, imaging, or treating diseases characterized by FAP expression, particularly by abnormal expression (e.g. overexpression, or expression in a different pattern in a cell or tissue) of FAP compared to normal tissue of the same cell type. FAP is abnormally expressed (e.g. overexpressed) in many human tumors compared to non-tumor tissue of the same cell type. Thus, the FRET constructs of this disclosure are particularly useful in the detection, imaging, and treatment of tumors. The FRET constructs of this disclosure can be used to treat any tissue, including tumor tissue, expressing FAP and having FAP enzymatic activity. Particular malignancies that can be diagnosed and then treated using the FAP constructs of this disclosure include, for example, lung cancer, colon cancer, gastric cancer, breast cancer, head and neck cancer, skin cancer, liver cancer, kidney cancer, prostate cancer, pancreatic cancer, brain cancer, cancer of the skeletal muscle.

Fibrosis-associated diseases or disorders that may be detected or diagnosed using a FRET construct of this disclosure include fibrotic liver diseases, such as nonalcoholic steatohepatitis (NASH), alcoholic steatohepatitis (ASH), viral hepatitis (Hepatitis virus), alcoholic liver disease, fatty liver disease, primary biliary cirrhosis, primary sclerosing cholangitis, alpha-1 antitrypsin deficiency, hemochromatosis, Wilson disease, autoimmune hepatitis, and cirrhosis.

Additionally, non-hepatic fibrotic diseases that may be detected or diagnosed using a FRET construct of this disclosure include chronic pancreatitis, cystic fibrosis, idiopathic pulmonary fibrosis, radiation-induced lung injury, atrial fibrosis, endomyocardial fibrosis, myocardial infarction, glial scar, arterial stiffness, arthrofibrosis, Crohn's disease, mediastinal fibrosis, myelofibrosis, Peyronie's disease, nephrogenic systemic fibrosis, progressive massive fibrosis, retroperitoneal fibrosis, scleroderma, systemic sclerosis, rheumatoid arthritis, osteoarthritis, atherosclerosis, systemic lupus erythematosus, fibromyalgia, Sjogren's syndrome, antiphospholipid syndrome, myasthenia gravis, multiple sclerosis, and glomerulosclerosis.

These fibrosis-associated diseases or disorders may include an insulin resistance-related disease, such as Type 2 diabetes, or polycystic ovary syndrome.

The fibrosis-associated disease or disorder may also be a solid tumor, such as hepatocellular carcinoma, pancreatic ductal carcinoma, renal carcinoma, gastrointestinal carcinoma, ovarian carcinoma, breast carcinoma, lung carcinoma, colorectal carcinoma, prostate carcinoma, endometrial carcinoma, bladder carcinoma, kidney carcinoma, or a thyroid carcinoma.

In these methods, the biological sample collected from the subject may be whole blood, serum, plasma, synovial fluid, cells or tissues from brain, breast, colon, kidney, liver, lung, ovary, pancreas, prostate, skeletal muscle, skin, small intestine, stomach, or uterus.

Exemplary methods of using the FRET constructs of this disclosure for imaging, include the imaging a FAP-expressing tissue in a subject, by administering to the subject a FRET construct of this disclosure, and detecting fluorescence from the molecular construct by in vivo imaging. In these imaging methods, the FRET construct may comprise near-infrared fluorescence (NIRF) fluorophores. In these methods, the in vivo imaging may be conducted using NIRF imaging, fluorescence reflectance imaging (FRI), fluorescence-mediated tomography (FMT), or any combination thereof. In these imaging techniques, the FAP-expressing tissue may be a solid tumor, and the method may further comprise resecting the tumor after the in vivo imaging. Similarly, the FAP-expressing tissue may be a fibrotic tissue within an organ, and the method may further comprise resecting the tumor fibrotic part of an organ after the in vivo imaging.

In related imaging methods of this disclosure, a FAP-expressing tissue in a subject is imaged by administering to the subject a FRET construct of this disclosure, and detecting fluorescence from the FRET construct by ex vivo imaging. The ex vivo imaging may comprise low resolution imaging of tissues excised from the subject. Alternatively or additionally, the ex vivo imaging may comprise in situ zymography of tissue slices from the subject.

In another aspect, a FRET construct of this disclosure for use as a medicament is provided. In further aspects, a FRET construct of this disclosure for use in treating a disease characterized by expression of FAP, or FAP enzymatic activity is provided. A FRET construct of this disclosure for use in a method of treatment is also provided.

In a further aspect, this disclosure provides for the use of a FRET construct of this disclosure in the manufacture or preparation of a medicament. The medicament may be used for detection or diagnosis of a disease characterized by expression of FAP or FAP enzymatic activity.

All patent and literature references cited in the present specification are hereby incorporated by reference in their entirety.

### Examples

### A. Experimental Procedures

Commercially available reagents referred to in these examples were used according to manufacturer's instructions unless otherwise indicated. The source of certain reagents are described below:

Recombinant Proteins Referred to in these Examples—

Human prolyl oligopeptidase (PREP, 4308-SE), DPPIV (1180-SE), DPP9 (5419-SE), and DPPII/DPP7 (3438-SE) proteins were purchased from R&D Systems. Human DPP8 (BML-SE527) protein was purchased from Enzo Life Sciences. N-terminal octa-His-tagged human and mouse FAP proteins containing the extracellular domain (residues L26-D760 and L26-D761, respectively) were purified from the conditioned media of transiently-transfected CHO cells, by immobilized metal affinity chromatography (IMAC), followed by size exclusion chromatography (SEC).

Inhibitors Referred to in these Examples—

The FAP-specific inhibitor (S)—N-(2-cyano-4,4-difluoropyrrolidin-1-yl)-2-oxoethyl)-quinoline-4-carboxamide (compound 60 or cpd60) was synthesized according to the experimental procedure reported by Jansen et al (J Med Chem 57:3053-74). The PREP-specific inhibitor KYP-2047 was purchased from Sigma-Aldrich (St. Louis, Mo.).

In Vivo Studies—

Mice were maintained in a pathogen-free animal facility at 21° C. under standard 12 h light/12 h dark cycle with access to normal chow (Labdiet 5010) and water ad libitum unless otherwise indicated. Generation of Fap KO mice was described previously (Dunshee, et al. (2016) J Biol Chem 291:5986-96). For in vivo pharmacological FAP inhibition, C57BL/6 background mice were fed ad libitum control chow (TD.00588, Envigo, Madison, Wis.), or the same diet containing 100 ppm of cpd60.

Plasma and Serum—

Mouse plasma samples were prepared in K₃-EDTA MiniCollect tubes (Greiner Bio-One, 450475) and frozen immediately at −80° C. prior to analysis. Human serum samples were obtained from healthy donors or purchased from Discovery Life Sciences, Inc. (Los Osos, Calif.).

FAP ELISA—

Serum FAP concentration was measured using the human FAP DuoSet ELISA kit (R&D Systems, DY3715), according to the manufacturer's protocol.

Fluorescence Resonance Energy Transfer (FRET)-Quench Assay—

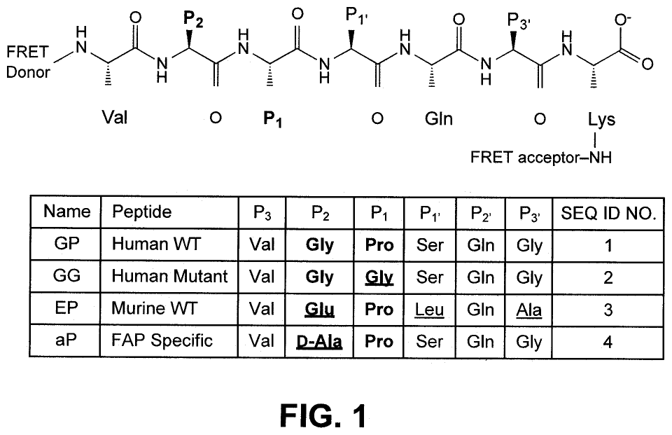

Peptides of the region flanking the C-terminal human FGF21 cleavage site (VGPSQG) or a variant with D-alanine at P2 (VaPSQG) were synthesized (Anaspec, Inc.), containing an amine-terminal donor (HyLite Fluor 488 or Cy5.5 for aP NIRF) and a dark quencher-acceptor (QXL 520 or QSY21 for aP NIRF) conjugated to a supplementary C-terminal lysine. Additional peptides were generated as above, with the residue sequences shown in FIG. 1. ANP_{FAP} (Bioconjugate Chem 23:1704-11) and aP NIRF were synthesized by CPC Scientific (Sunnyvale, Calif.). Assays were conducted at 37° C. in 50 mM HEPES (pH 7.2), 150 mM NaCl, 1 mM EDTA, 0.1 mg/ml BSA. The excitation/emission wavelengths of cleaved peptide are 490/520 nm or 675/695 nm for ANP_{FAP} and aP NIRF. Fluorescence was monitored on a Tecan M1000 Pro plate reader in kinetic mode. Except where indicated otherwise, measurements of serum or plasma FAP activity were performed with the peptide substrate concentration at 6 μM and a 10-fold dilution of serum or plasma in assay buffer.

Enzyme Kinetics—

Michaelis-Menten kinetic measurements were carried out using 5 nM human or mouse recombinant FAP, with a series of peptide substrate titrations. Initial rate of substrate hydrolysis was determined using the Magellan software on a Tecan M1000 Pro plate reader and kinetic parameters were modeled using nonlinear regression analysis with GraphPad Prism software. Standard error was calculated from three experimental replicates.

Active Site Titration—

The tight-binding FAP inhibitor, cpd60, was titrated into a GP peptide cleavage reaction with 100 nM of mouse or human recombinant FAP and 6 μM of GP. The IC₅₀ of cpd60 for human and mouse FAP endopeptidase activity was previously determined to be <0.5 nM (Dunshee, et al. (2016) J Biol Chem 291:5986-96). As described by Copeland ((2000) Enzymes: a practical introduction to structure, mechanism, and data analysis, 2nd ed., J. Wiley, New York) the active enzyme concentration was determined from the x-intercept.

Statistics—

Unpaired student's t-test (two-tailed) was used for statistical analysis to compare treatment groups. A p value <0.05 was considered statistically significant. All the values were presented as means±SEM unless otherwise noted.

### B. Results

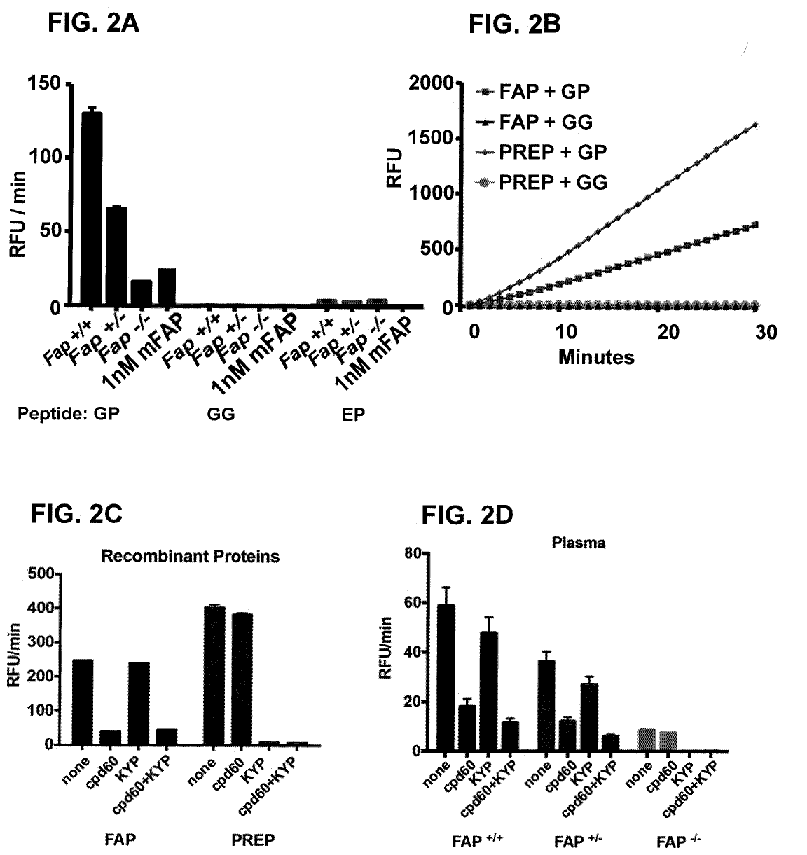

An FGF21-based Quenched-Fluorescence peptide is cleaved by FAP and PREP. A peptide containing the six amino acid residues surrounding the FAP cleavage site near the C-terminus of human FGF21, termed the “GP” probe, (FIG. 1) was synthesized for the purpose of monitoring FAP endopeptidase activity. The peptide is flanked by a FRET-donor (HyLite Fluor 488) and a dark quencher (QXL 520). By design, fluorescence intensity is suppressed due to close proximity of the quencher dye to the donor fluorophore, and it is liberated by protease-catalyzed cleavage of the peptide. As controls, variant peptides containing a substitution of the P1 proline with glycine (“GG” probe) or the homologous region of murine FGF21 (“EP” probe) were also generated. Both control probes lack the Gly-Pro consensus necessary for FAP-based cleavage, thus serve as negative controls (FIG. 1). These three peptides were used to evaluate the FAP endopeptidase activity in the plasma of wild type (WT), heterozygous and Fap-deficient (KO) mice. We have previously demonstrated by immunoblot analysis that FAP is present in the plasma from WT mice, to a lesser degree in heterozygotes and undetectable in KO mice (Dunshee, et al. (2016) J Biol Chem 291:5986-96). When the GP probe was used, plasma from WT mice produced a strong signal, indicating efficient cleavage of the probe (FIG. 2A). As expected, plasma from Fap heterozygous mice exhibited approximately half the activity as that from WT mice, and that from homozygous Fap KO mice exhibited even lower, but significant activity. When plasma from WT mice was tested using GG and EP probes, no and a very minor signal was obtained respectively. Furthermore, purified recombinant mouse FAP protein cleaved the GP but not GG or EP probes (FIG. 2A).

The ability of Fap KO mouse plasma to cleave the GP probe and to a much lesser degree, the EP probe, but not the GG probe, suggested the presence of other proline-specific endopeptidases in plasma. Although prolyl endopeptidase (PREP) is predominantly a cytosolic enzyme, it is known to be present at variable levels in plasma, and seemed a likely candidate for the source of residual probe cleavage by the KO plasma. Indeed, when 1 nM of recombinant human FAP or PREP was tested, both enzymes readily cleaved the GP peptide, while leaving the mutant GG peptide intact (FIG. 2B). To further investigate the involvement of PREP in the cleavage of the GP probe, we used a FAP-selective inhibitor, cpd60 (Dunshee, et al. (2016) J Biol Chem 291:5986-96) and a PREP-selective inhibitor KYP-2047 (Venalainen, et al., (2000) Biochemical Pharmacology 71:683-92). Both inhibitors were tested at a concentration that provides selective inhibition of each enzyme (FIG. 2C). Using these inhibitors, we found that the residual GP probe cleaving activity in Fap KO sample was sensitive to KYP, but not cpd60 (FIG. 2D). At the same time, the GP probe activity in WT or heterozygous samples were clearly sensitive to cpd60. Taken together, we concluded that FAP is the major enzyme responsible for the cleavage of the GP probe in plasma, while PREP has a smaller but significant contribution.

Impact of P₂ D-Ala Substitution on FAP Cleavage Kinetics.

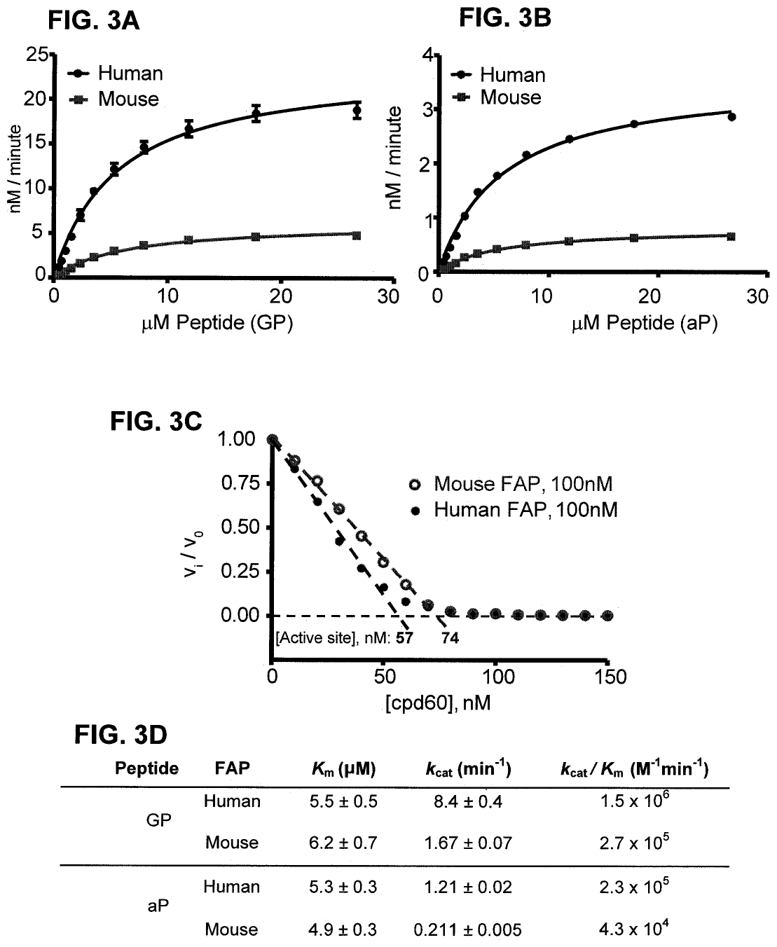

FAP has previously been shown to tolerate a change of the consensus glycine to a D-alanine at position P₂ in a α2AP-based peptide substrate, although a weaker catalytic efficiency was observed relative to the parental peptide due to a decrease in k_{cat} of approximately eight-fold. In the context of FAP inhibitors, this substitution has been reported to impart specificity for FAP over PREP (Poplawski, et al (2013) J Med Chem 56, 3467-377). In an attempt to increase the specificity of our assay, we incorporated this substitution into the FGF21-based GP peptide to produce the “aP” probe (FIG. 1). For an activity probe to be of practical use for a diagnostic purpose, it is important that this change does not have a strong negative impact on the enzyme kinetics. We therefore compared the Michaelis-Menten kinetics for both the GP and aP peptides, using both human and mouse FAP proteins (FIGS. 3A, 3B & 3D).

Interestingly, mouse FAP exhibited a lower catalytic efficiency than human FAP, mainly due to a reduced k_{cat}(FIGS. 3A & 3B). From the kinetics data alone, it was not clear if this is an innate efficiency difference between the orthologs or if it was due to differences in active enzyme concentration between the two preparations. To address this question, an active site titration was performed, using the tight binding inhibitor, cpd60 (FIG. 3C). We found that the preparation of mouse FAP had, in fact, a higher relative concentration of active enzyme, confirming its intrinsically weaker catalytic efficiency. The kinetics data shown in FIG. 3D are thus corrected for the experimentally determined active enzyme concentration. The Kₘ value is essentially unchanged between the parental (GP) and aP peptides, and we observed the expected seven to eight-fold decrease in k_{ca}t for aP cleavage.

P₂ D-Ala Substitution Provides FAP Specificity.

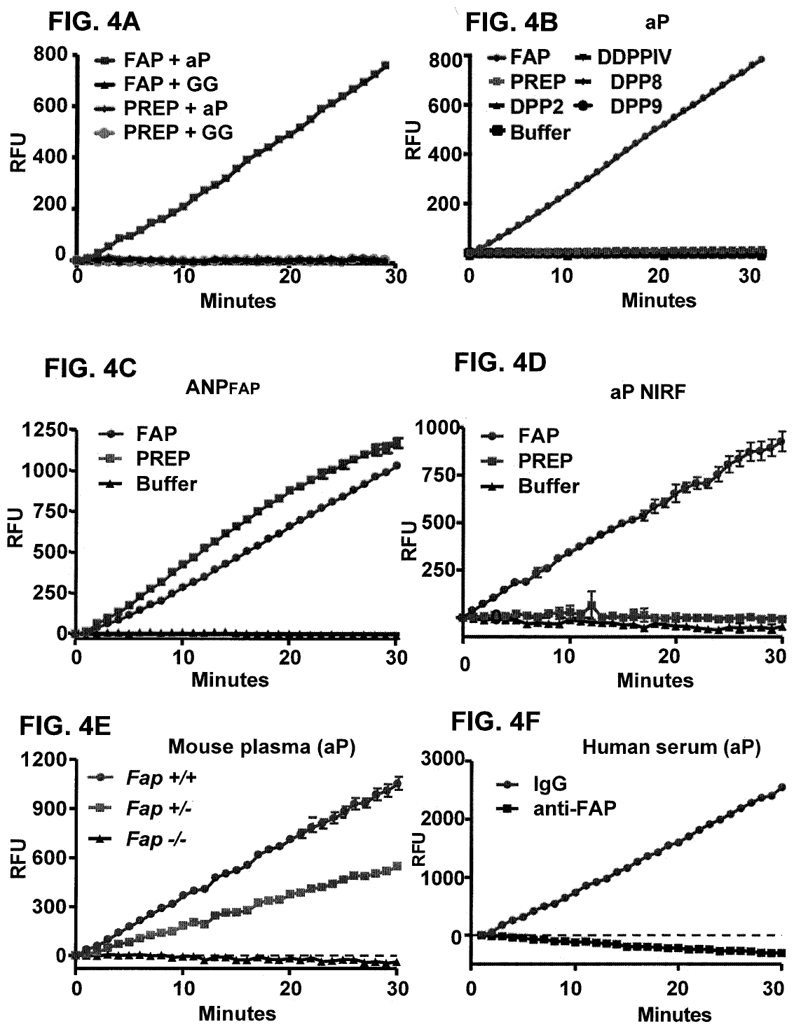

Encouraged by the kinetics data, we decided to test the specificity of the aP probe against PREP and a panel of related peptidases. Using a concentration of 1 nM enzyme, cleavage of the aP probe by human FAP is clearly detectable, but not the GG probe (FIG. 4A). In contrast, human PREP is unable to digest either probe. In addition, a broader set of human S9 and S28 peptidases, did not exhibit an ability to cleave the aP probe (FIG. 4B). This is consistent with the general lack of endopeptidase activity of these prolyl dipeptidyl peptidases.

Another internally-quenched FRET peptide substrate (ANP_{FAP}) for FAP has been reported and demonstrated for use as an activity-based, in vivo imaging tool (Li, et al. (2012) Bioconjugate Chem 23, 1704-1711). The peptide sequence contains two internal Gly-Pro dipeptide motifs, susceptible to FAP cleavage and a Cy5.5/QSY21, quenched-FRET pair. To evaluate specificity, the authors tested ANP_{FAP} for cleavage in vitro by FAP, DPPIV and MMP-2, but only detected cleavage in the presence of FAP. Our experience with the GP probe led us to suspect that the Gly-Pro motifs in ANP_{FAP} are also recognized by PREP. Therefore, ANP_{FAP} was synthesized and tested for cleavage by recombinant human FAP and PREP (FIG. 4C). As suspected, both FAP and PREP efficiently cleave ANP_{FAP} in vitro. While it is currently unclear how much of a liability PREP or other non-specific prolyl endopeptidase activity might be in the context of in vivo FAP imaging, we were interested to see if our aP probe would retain its specificity and FAP cleavability if the FRET pair were replaced with near infrared fluorophores (NIRF) more suitable to in vivo imaging. A new peptide was generated (aP NIRF), identical to aP, but the FRET donor and quencher were replaced with Cy5.5 and QSY21, respectively. In contrast to ANP_{FAP}, we found that FAP, but not PREP, efficiently cleaves aP NIRF (FIG. 4D).

Finally, having demonstrated specificity using several purified recombinant enzymes, we returned to the analysis of the WT and Fap KO mice plasma as in FIG. 2A, but using the aP probe. As expected, the aP probe shows a clear signal with WT plasma, intermediate with heterozygous plasma and satisfyingly, the aP probe exhibits no cleavage with Fap KO plasma samples (FIG. 4E). Furthermore, signal is lost after FAP immunodepletion from human serum, but not in control IgG-immunodepleted serum (FIG. 4F).

The Diagnostic Utility of aP Probe in Analyzing FAP Activity in Blood.

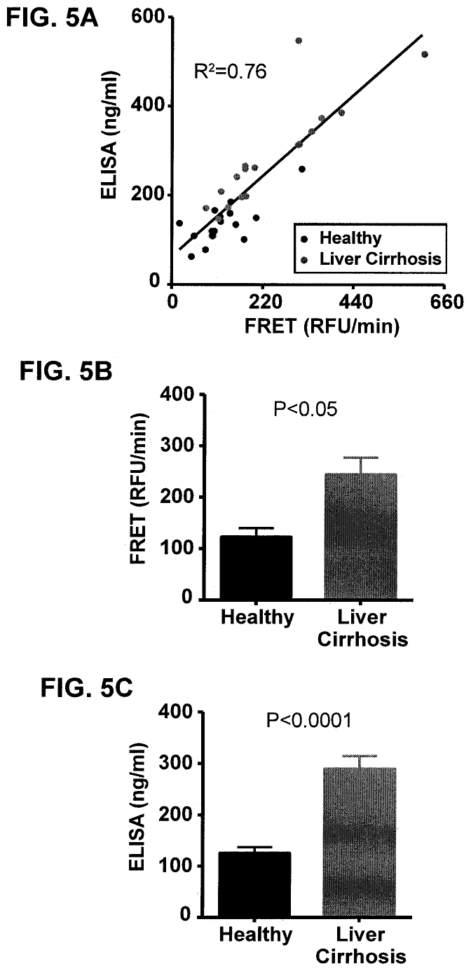

Previously, serum FAP levels and activity have been shown to correlate with liver fibrosis. Therefore, we decided to determine if we can recapitulate these findings by the aP probe-based fluorescence assay. We obtained serum samples from healthy volunteers and from individuals who had been diagnosed with non-viral liver cirrhosis. When these two groups were compared, we found a statistically significant increase in FAP activity (by fluorescent intensity assay with aP probe) (FIGS. 5A and 5B) and in FAP levels (by a conventional sandwich ELISA) (FIGS. 5A and 5C). The observed FAP activity correlated well with FAP protein level (R²=0.76, p values <0.05) and using the activity assay, we were easily able to detect levels as low as 63 ng/ml (FIG. 5A).

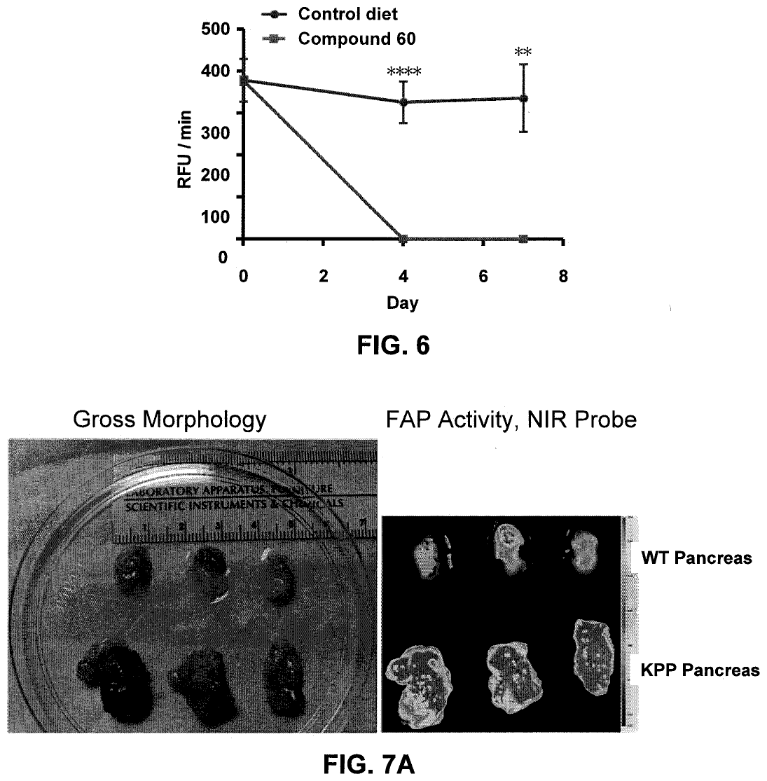

We next wanted to determine whether the new aP-based assay can be used to determine the pharmacodynamic activity of FAP inhibitors in vivo. To do this, we fed mice with chow containing the FAP inhibitor cpd60 or a control diet without the inhibitor (FIG. 6). The FAP activity was determined in plasma isolated at day 4 and 7 after cpd60 feeding started. The results clearly showed that cpd60 feeding could completely suppress circulating FAP activity in mice. Therefore, we conclude that the aP-based assay can be used to evaluate the pharmacodynamics of FAP inhibitors selectively, without interference from PREP activity potentially present in plasma.

The Diagnostic Utility of a NIRF Probe in Analyzing FAP Activity in Tissue.

The utility of the VaPSQG-NIRF probe for imaging of tissue FAP activity was studied in a genetically engineered mouse model of pancreatic cancer, KrasLSL.G12D/wt;p16/p19fl/fl;Pdx1. Cre, or “KPP” for short (Singh, M. et al. Nat. Biotechnol. 28:585-593 (2010); Aguirre, A. J. et al. Genes Dev. 17:3112-26 (2003)). The KPP mouse strain has been shown to spontaneously develop ultrasound-detectable pancreatic tumors. A similar mouse strain (KrasLSL.G12D/wt; Tp53LSL.R172H/wt; Pdx1.Cre) has been shown to express FAP in pancreatic tumor stroma (Feig, C. et al., Proc Natl Acad Sci USA. 110:20212-17 (2013).). The VaPSQG-NIRF probe was synthesized by CPC Scientific (Sunnyvale, Calif.).

Prior to the imaging study, three KPP mice (9.3 weeks old) with varying tumor volumes were selected based on an in vivo ultrasound scan. Additionally, three healthy C57BL/6 mice (8.3 weeks old) were also studied as controls. One nmol VaPSQG-NIRF probe diluted in 150 microL of PBS was intravenously administered to each mouse via tail vein under anesthesia. No treatment-related adverse effect was observed between the probe administration and the euthanasia. After 4 hours, the mice were euthanized and pancreas tissues were dissected. The dissected tissues were rinsed with PBS, dried with tissue paper, and put on black paper for ex vivo NIRF imaging. Near infrared images are acquired using a CCD camera (Pearl Trilogy, Li-cor).

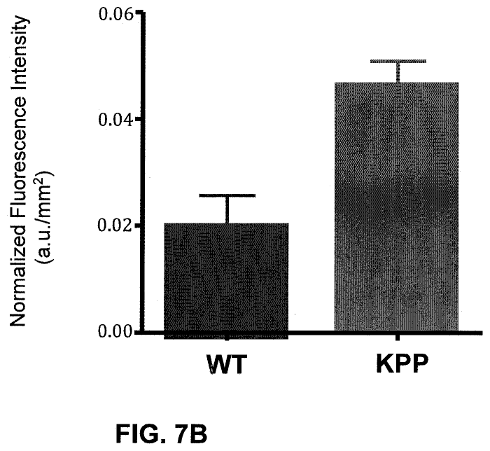

After successfully validating the specificity of aP NIRF in vitro (FIG. 4D), its potential use for ex vivo imaging was investigated. For this purpose we used the KPP mouse strain, which expresses FAP in the pancreatic tumor stroma, and healthy C57BL/6 mice were included as controls. Ex vivo images of three pancreata from each group are shown in FIG. 7A, with the gross morphology shown in the left panel and a heatmap representation of the aP NIRF probe imaging signal intensity in the right panel. The KPP group (bottom) had enlarged pancreata relative to the healthy control group and exhibited a strong fluorescent signal. Quantification of the relative fluorescence intensities normalized to the imaging area (FIG. 7B) shows a much greater fluorescence in the KPP pancreata (P<0.05), demonstrating that aP NIRF can be successfully employed for ex vivo imaging of FAP.

### C. Discussion

These data demonstrate that the new FRET assay of this disclosure, based on an FGF21 peptide containing the D-Ala-Pro dipeptide, provides several notable advancements. First and foremost, the assay enables the measurement of FAP endopeptidase activity in serum or plasma samples without immunocapture or any other purification steps, even for samples that contain related S9 proteases, most notably PREP. Second, because of its strict specificity, the activity assay for FAP will substitute for the more cumbersome sandwich ELISA, at least for certain applications. Indeed, we observed a very good correlation between FAP activity and FAP protein levels in human serum samples from both healthy individuals and patients with cirrhosis (FIG. 5). This will save time and cost for investigators interested in determining FAP levels in samples that do not contain FAP inhibitors. Finally, the aP probe can be synthesized from commonly used fluorescent dyes and amino acids without any specialized methods. This means the probe can be ordered from a peptide probe vendor through a custom synthesis request. This increases the accessibility, practicality and utility of the new assay methods of this disclosure.

As a practical application of the new assay, we demonstrated that a FAP-selective inhibitor, cpd60, can be used in vivo to suppress the activity of circulating FAP in a sustained fashion in mice. We previously tested this compound in cynomolgus monkeys via bolus p.o. administration and achieved a transient decrease in FAP-related activity, consistent with the rapid clearance of this compound from circulation (Dunshee, et al. (2016) J Biol Chem 291:5986-96). In this study, therefore, we tested the strategy of feeding cpd60-containing chow to mice and used the aP probe to track the resulting pharmacodynamics. The results indicated that cpd60 suppressed FAP activity in mice to undetectable levels. The ability of the aP probe to monitor changes in FAP activity without an influence of the residual PREP activity allowed us to demonstrate that cpd60 could completely suppress FAP at the dose we tested. This feeding-based approach will facilitate pre-clinical investigation of the role of FAP to regulate endopeptidic substrates, such as α2AP and FGF21, as well as its role in diseases for which FAP has been implicated.

In conclusion, we have developed a unique, accessible and specific homogenous assay for the endopeptidase activity of FAP. The assay disclosed here will facilitate preclinical investigations of FAP biology and the clinical development of FAP inhibitors or FAP-localized anti-tumor therapies in the future.

The present disclosure is not to be limited in scope by the specific embodiments described herein which are intended as single illustrations of individual aspects of this disclosure, and functionally equivalent methods and components are within the scope of this disclosure. Indeed, various modifications of this disclosure, in addition to those shown and described herein will become apparent to those skilled in the art from the foregoing description and accompanying drawings. Such modifications are intended to fall within the scope of the claims.

What is claimed is:

1. A molecular fluorescence resonance energy transfer (FRET) construct comprising a linker peptide, a donor fluorophore moiety and an acceptor fluorophore moiety, wherein the linker peptide is a substrate of Fibroblast Activation Protein (FAP) endopeptidase.

2. The molecular construct of claim 1, wherein the linker peptide is not a substrate for a human S9 peptidase.

3. The molecular construct of claim 1, wherein the linker peptide is not a substrate for a human S28 peptidase.

4. The molecular construct of claim 1, wherein the linker peptide is not a substrate for any one of dipeptidyl peptidase IV (DPPIV), DPP8, or DPP9.

5. The molecular construct of claim 1, wherein the linker peptide is not a substrate for prolyl endopeptidase (PREP).

6. The molecular construct of claim 1, wherein the linker peptide separates the donor and acceptor fluorophores by a distance of not more than 10 nm, and wherein absorption spectrum of the donor fluorophore moiety overlaps with the excitation spectrum of the acceptor fluorophore moiety.

7. The molecular construct of claim 1, wherein the linker peptide is a peptide comprising the sequence:

| P3 | P2 | P1 | P1′ | P2′ | P3′ | |
|---|---|---|---|---|---|---|
| Xaa1- | Xaa2- | Xaa3- | Xaa4- | Xaa5- | Xaa6- | (SEQ ID |
| | | | | | | NO: 1) |

wherein Xaa1 is: a natural or non-natural amino acid residue or derivative;
wherein Xaa2 is: a residue selected from the D-enantiomer of a naturally occurring amino acid, or an amino acid analog;
wherein Xaa3 is: proline (Pro) or a derivative thereof;
wherein Xaa4 is: a natural or non-natural amino acid residue or derivative;
wherein Xaa5 is: a natural or non-natural amino acid residue or derivative; and,
wherein Xaa6 is: a natural or non-natural amino acid residue or derivative.

8. The molecular construct of claim 7, wherein
Xaa2 is selected from D-alanine, D-serine, and D-threonine.

9. The molecular construct of claim 7, wherein
Xaa2 is an amino acid analog having the formula —NH—(CH₂)ₙ—COO— wherein n=2-10.

10. The molecular construct of claim 9, wherein
Xaa2 is an amino acid analog selected from beta-alanine (bAla), gamma-aminobutryic acid (4Abu), 5-aminovaleric acid, and 6-aminohexanoic acid.

11. The molecular construct of claim 7, wherein
Xaa3 is a halogenated proline residue.

12. The molecular construct of claim 11, wherein
Xaa3 is a fluorinated proline residue.

13. The molecular construct of claim 7, wherein
Xaa3 is a derivative of proline selected from dehydroproline, 4,4-difluoroproline, 3-fluroproline, 4-fluroproline, 3-hydroxyproline (3Hyp), and 4-hydroxyproline (4Hyp).

14. The molecular construct of claim 7, wherein Xaa1 is covalently linked to either the donor fluorophore moiety or the acceptor fluorophore moiety.

15. The molecular construct of claim 7, wherein Xaa6 is covalently linked to either the donor fluorophore moiety or the acceptor fluorophore moiety.

16. The molecular construct of claim 7, wherein at least one of the donor fluorophore moiety and the acceptor fluorophore moiety are covalently linked to the linker peptide through a linker.

17. The molecular construct of claim 16, wherein the linker comprises at least one amino acid selected from lysine (Lys), arginine (Arg), glutamine (Gln), and asparagine (Asn).

18. The molecular construct of claim 16, wherein the linker is a lysine (Lys) residue.

19. The molecular construct of claim 7, wherein at least one of the donor fluorophore moiety and the acceptor fluorophore moiety are linked to the amino-terminus of the peptide.

20. The molecular construct of claim 19, wherein at least one of the donor fluorophore moiety and the acceptor fluorophore moiety are linked to the carboxy-terminus of the peptide.

21. The molecular construct of claim 19, wherein the donor fluorophore moiety is linked to the carboxy-terminus of the peptide and the acceptor fluorophore moiety is linked to the amino-terminus of the peptide.

22. The molecular construct of claim 19, wherein the donor fluorophore moiety is linked to the amino-terminus of the peptide and the acceptor fluorophore moiety is linked to the carboxy-terminus of the peptide.

23. The molecular construct of claim 7, wherein the peptide comprises or consists of a peptide having the sequence Val-(D-Ala)-Pro-Ser-Gln-Gly (SEQ ID NO:2).

24. The molecular construct of claim 23, comprising a peptide with at least 80% amino acid sequence identity to the amino acid sequence of SEQ ID NO:2, and wherein the peptide is a substrate for FAP but is not a substrate for a human S9 or S28 peptidase.

25. The molecular construct of claim 23, comprising a peptide with at least 80% amino acid sequence identity to the amino acid sequence of SEQ ID NO:2, and wherein the peptide is a substrate for FAP but is not a substrate for a human S9 or S28 peptidase.

26. The molecular construct of claim 23, comprising a peptide having between 1 and 3 conservative amino acid substitutions compared to the amino acid sequence of SEQ ID NO:2, wherein the peptide is a substrate for FAP but is not a substrate for a human S9 or S28 peptidase.

27. The molecular construct of claim 23, comprising a peptide having one conservative amino acid substitutions compared to the amino acid sequence of SEQ ID NO:2, wherein the peptide is a substrate for FAP but is not a substrate for a human S9 or S28 peptidase.

28. The molecular construct of claim 7, wherein the peptide comprises a modification selected from phosphorylation and glycosylation.

29. The molecular construct of claim 7, wherein the peptide is linked to a polyethylene glycol (PEG) molecule.

30. The molecular construct of claim 29, wherein the number of ethylene glycol (EG) units in the PEG molecule is between 75 to 2000.

31. The molecular construct of claim 7, wherein the peptide is linked to one or more domains of an Fc region of human IgG molecule.

32. The molecular construct of claim 31, wherein the Fc region is a human IgG hinge, CH2, and CH3 region that is fused to at least one of the amino-terminus or carboxyl-terminus of the peptide.

33. The molecular construct of claim 7, wherein the peptide is linked to an epitope tag polypeptide comprising between 6 and 50 amino acid residues.

34. The molecular construct of claim 7, which is linked to a solid support.

35. The molecular construct of claim 34, wherein the solid support comprises at least one of glass, polysaccharides, polyacrylamides, polystyrene, polyvinyl alcohol, and silicones.

36. A nucleic acid molecule encoding at least one of the peptides of claims 1-27.

37. An expression vector comprising the nucleic acid molecule of claim 36 operably linked to a control sequence for the expression of the peptide of any one of claims 1-27.

38. A host cell comprising the expression vector of claim 37.

39. A composition comprising a molecular construct of any one of claims 1-35, and at least one pharmaceutically acceptable excipient.

40. A method of determining fibroblast activation protein (FAP) enzymatic activity in a sample, comprising:
a) obtaining a biological sample from a mammalian subject;
b) detecting whether FAP enzymatic activity is present in the sample by contacting at least a portion of the sample with a molecular construct of any one of claims 1-35, illuminating the sample, and detecting fluorescence resulting from FAP cleavage of the peptidase indicator construct; and
c) determining the FAP enzymatic activity in the sample by comparing the fluorescence resulting from FAP cleavage of the molecular construct with a reference correlation of fluorescence and FAP enzymatic activity.

41. The method of claim 40, wherein the biological sample is whole blood, serum, plasma, synovial fluid, cells or tissues or lysates thereof, cell culture supernatant, or a sample comprising a recombinant FAP protein.

42. The method of claim 40 or 41, further comprising:
d) contacting a second portion of the sample with the molecular construct and a putative inhibitor of FAP enzymatic activity, illuminating the sample, and detecting fluorescence resulting from FAP cleavage of the molecular construct; and
e) determining the FAP enzymatic activity in the sample by comparing the fluorescence resulting from FAP cleavage of the molecular construct with a reference correlation of fluorescence and FAP enzymatic activity
f) calculating the inhibition of FAP enzymatic activity resulting from the molecular inhibitor of FAP enzymatic activity as the FAP enzymatic activity in the second portion of the sample subtracted from the FAP enzymatic activity in the portion of the sample.

43. A method of diagnosing a fibrosis-associated disease or disorder in a subject, comprising:
a) obtaining a biological sample from a mammalian subject;
b) detecting whether FAP enzymatic activity is present in the sample by contacting a portion of the sample with a molecular construct of any one of claims 1-35, illuminating the sample, and detecting fluorescence resulting from FAP cleavage of the peptide construct; and
c) diagnosing the subject with fibrosis-associated disease or disorder when fluorescence resulting from FAP enzymatic activity is detected in the sample.

44. The method of claim 43, wherein the diagnosing comprises determining a level of FAP enzymatic activity in the sample by comparing the fluorescence resulting from FAP cleavage of the molecular construct to a reference level of FAP enzymatic activity in the fibrosis-associated disease or disorder, wherein a statistically equal or higher level of FAP enzymatic activity in the sample compared to the reference level is indicative of a fibrosis-associated disease or disorder in the subject.

45. The method of claim 43 or 44, wherein the biological sample is whole blood, serum, plasma, synovial fluid, cells, or tissues.

46. The method of any one of claims 43-45, wherein the fibrosis-associated disease or disorder is a fibrotic liver disease.

47. The method of claim 46, wherein the fibrotic liver disease is selected from nonalcoholic steatohepatitis (NASH), alcoholic steatohepatitis (ASH), viral hepatitis (Hepatitis virus), alcoholic liver disease, fatty liver disease, primary biliary cirrhosis, primary sclerosing cholangitis, alpha-1 antitrypsin deficiency, hemochromatosis, Wilson disease, autoimmune hepatitis, and cirrhosis.

48. The method of any one of claims 43-45, wherein the fibrosis-associated disease or disorder is a non-hepatic fibrotic disease.

49. The method of claim 48, wherein the non-hepatic fibrotic disease is selected from chronic pancreatitis, cystic fibrosis, idiopathic pulmonary fibrosis, radiation-induced lung injury, atrial fibrosis, endomyocardial fibrosis, myocardial infarction, glial scar, arterial stiffness, arthrofibrosis, Crohn's disease, mediastinal fibrosis, myelofibrosis, Peyronie's disease, nephrogenic systemic fibrosis, progressive massive fibrosis, retroperitoneal fibrosis, scleroderma, systemic sclerosis, rheumatoid arthritis, osteoarthritis, atherosclerosis, systemic lupus erythematosus, fibromyalgia, Sjogren's syndrome, antiphospholipid syndrome, myasthenia gravis, multiple sclerosis, and glomerulosclerosis.

50. The method of any one of claims 43-45, wherein the fibrosis-associated disease or disorder is an insulin resistance-related disease.

51. The method of claim 50, wherein the insulin resistance-related disease is Type 2 diabetes or polycystic ovary syndrome.

52. The method of any one of claims 43-45, wherein the fibrosis-associated disease or disorder is a solid tumor.

53. The method of claim 52, wherein the tumor is selected from a hepatocellular carcinoma, pancreatic ductal carcinoma, renal carcinoma, gastrointestinal carcinoma, ovarian carcinoma, breast carcinoma, lung carcinoma, colorectal carcinoma, prostate carcinoma, endometrial carcinoma, bladder carcinoma, kidney carcinoma, and a thyroid carcinoma.

54. A method of diagnosing and treating a fibrosis-associated disease or disorder in a subject, comprising:
a) obtaining a biological sample from a human subject;
b) detecting whether FAP enzymatic activity is present in the sample by contacting a portion of the sample with a molecular construct of any one of claims 1-35, illuminating the sample, and detecting fluorescence resulting from FAP cleavage of the peptide construct; and,
c) diagnosing the subject with a fibrosis-associated disease or disorder by comparing the fluorescence resulting from FAP cleavage of the molecular construct to a reference level of FAP enzymatic activity in the fibrosis-associated disease or disorder, wherein a statistically equal or higher level of FAP enzymatic activity in the sample compared to the reference level is indicative of a fibrosis-associated disease or disorder in the subject; and,
d) administering an effective therapy to the diagnosed subject.

55. The method of claim 54, wherein the fibrosis-associated disease or disorder is a fibrotic liver disease.

56. The method of claim 55, wherein the fibrotic liver disease is selected from nonalcoholic steatohepatitis (NASH), alcoholic steatohepatitis (ASH), viral hepatitis (Hepatitis virus), alcoholic liver disease, fatty liver disease, primary biliary cirrhosis, primary sclerosing cholangitis, alpha-1 antitrypsin deficiency, hemochromatosis, Wilson disease, autoimmune hepatitis, and cirrhosis.

57. The method of any claim 54, wherein the fibrosis-associated disease or disorder is a non-hepatic fibrotic disease.

58. The method of claim 57, wherein the non-hepatic fibrotic disease is selected from chronic pancreatitis, cystic fibrosis, idiopathic pulmonary fibrosis, radiation-induced lung injury, atrial fibrosis, endomyocardial fibrosis, myocardial infarction, glial scar, arterial stiffness, arthrofibrosis, Crohn's disease, mediastinal fibrosis, myelofibrosis, Peyronie's disease, nephrogenic systemic fibrosis, progressive massive fibrosis, retroperitoneal fibrosis, scleroderma, systemic sclerosis, rheumatoid arthritis, osteoarthritis, atherosclerosis, systemic lupus erythematosus, fibromyalgia, Sjogren's syndrome, antiphospholipid syndrome, myasthenia gravis, multiple sclerosis, and glomerulosclerosis.

59. The method of claim 54, wherein the fibrosis-associated disease or disorder is an insulin resistance-related disease.

60. The method of claim 59, wherein the insulin resistance-related disease is Type 2 diabetes or polycystic ovary syndrome.

61. The method of claim 54, wherein the fibrosis-associated disease or disorder is a solid tumor.

62. The method of claim 61, wherein the tumor is selected from a hepatocellular carcinoma, pancreatic ductal carcinoma, renal carcinoma, gastrointestinal carcinoma, ovarian carcinoma, breast carcinoma, lung carcinoma, colorectal carcinoma, prostate carcinoma, endometrial carcinoma, bladder carcinoma, kidney carcinoma, and a thyroid carcinoma.

63. The method of any one of claims 54-62, wherein the biological sample is whole blood, serum, plasma, synovial fluid, cells or tissues from brain, breast, colon, kidney, liver, lung, ovary, pancreas, prostate, skeletal muscle, skin, small intestine, stomach, or uterus.

64. The method of any one of claims 54-62, wherein the therapy comprises an inhibitor of FAP enzymatic activity.

65. The method of any one of claims 54-62, wherein the therapy comprises an anti-FAP enzyme antibody.

66. The method of claim 65, wherein the anti-FAP enzyme antibody is a bi-specific antibody.

67. The method of claim 65, wherein the anti-FAP enzyme antibody is an antibody drug conjugate (ADC).

68. A method of imaging a FAP-expressing tissue in a subject, the method comprising:
a) administering to the subject a molecular construct of any one of claims 1-35; and
b) detecting fluorescence from the molecular construct by in vivo imaging.

69. The method of claim 68, wherein the molecular construct comprises near-infrared fluorescence (NIRF) fluorophores.

70. The method of claim 68 or 69, wherein the in vivo imaging is selected from the group consisting of NIRF imaging, fluorescence reflectance imaging (FRI), fluorescence-mediated tomography (FMT), and any combination thereof.

71. The method of any one of claims 68 to 70, wherein the FAP-expression tissue is a solid tumor and further comprising resecting the tumor after the in vivo imaging.

72. The method of any one of claims 68 to 70, wherein the FAP-expression tissue is a fibrotic tissue within an organ and further comprising resecting the tumor fibrotic part of an organ after the in vivo imaging.

73. A method of imaging a FAP-expressing tissue in a subject, the method comprising:
a) administering to the subject a molecular construct of any one of claims 1-35; and
b) detecting fluorescence from the molecular construct by ex vivo imaging.

74. The method of claim 73, wherein the ex vivo imaging comprises low resolution imaging with excised tissues.

75. The method of claim 73, wherein the ex vivo imaging comprises in situ zymography of tissue slices.
